(19) United States
(12) Patent Application Publication
(10) Pub. No.: US 2025/0177649 A1
(43) Pub. Date: Jun. 5, 2025

(54) SYSTEM AND METHOD FOR INTEGRATION OF INSULIN PUMPS AND CONTINUOUS GLUCOSE MONITORING

(71) Applicant: Tandem Diabetes Care, Inc., San Diego, CA (US)

(72) Inventors: Michael Rosinko, Anaheim, CA (US); Sean Saint, San Diego, CA (US); Jason Farnan, San Diego, CA (US); Shaun Buchanan, Carlsbad, CA (US)

(21) Appl. No.: 19/050,374

(22) Filed: Feb. 11, 2025

Related U.S. Application Data
(63) Continuation of application No. 18/108,902, filed on Feb. 13, 2023, now Pat. No. 12,251,536.
Application No. 18/108,902 is a continuation of application No. 16/590,836, filed on Oct. 2, 2019, now Pat. No. 11,607,492.
Application No. 16/590,836 is a continuation of application No. 15/072,408, filed on Mar. 17, 2016, now abandoned.
Application No. 15/072,408 is a continuation of application No. 13/800,453, filed on Mar. 13, 2013, now Pat. No. 10,357,606.

(51) Int. Cl. A61M 5/172; A61M 5/142; G16H 20/17

(52) U.S. Cl. CPC A61M 5/1723; A61M 5/14244; G16H 20/17; A61M 2205/502; A61M 2205/505; A61M 2205/52; A61M 2205/8206; A61M 2230/201

(57) ABSTRACT

A portable insulin pump can integrate and display data from a continuous glucose monitor (CGM) to allow a user to more readily determine whether any interaction with the pump is necessary. Data from the CGM can automatically be transmitted to the pump and can be displayed for user analysis or automatically analyzed to present recommendations to the user based on combined data from the CGM and the pump.

### RELATED APPLICATIONS

This application is a continuation of application Ser. No. 18/108,902, filed Feb. 13, 2023, continuation of application Ser. No. 16/590,836, filed Oct. 2, 2019, now U.S. Pat. No. 11,607,492, issued Mar. 21, 2023, which is a continuation of application Ser. No. 15/072,408, filed Mar. 17, 2016, which is a continuation of application Ser. No. 13/800,453 filed Mar. 13, 2013, now U.S. Pat. No. 10,357,606 issued Jul. 23, 2019, each of which is hereby fully incorporated herein by reference.

### FIELD OF THE INVENTION

The present invention relates to ambulatory infusions pumps and, more particularly, to integrating features of continuous glucose monitoring with insulin pumps.

### BACKGROUND

There are many applications in academic, industrial, and medical fields that benefit from devices and methods that are capable of accurately and controllably delivering fluids, such as liquids and gases that have a beneficial effect when administered in known and controlled quantities. Such devices and methods can be particularly useful in the medical field where treatments for many patients include the administration of a known amount of a substance at predetermined intervals.

Insulin-injecting pumps have been developed for the administration of insulin for those suffering from both type I and type II diabetes. Recently, continuous subcutaneous insulin injection and/or infusion therapy with portable infusion devices has been adapted for the treatment of diabetes. Such therapy may include the regular and/or continuous injection or of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. Patent Application No. 12/538,018, U.S. Provisional Patent Application No. 61/655,883, U.S. Provisional Patent Application No. 61/656,967 and U.S. Pat. No. 8,287,495, each of which is incorporated herein by reference.

Portable insulin pumps can be used in conjunction with continuous glucose monitoring (CGM) devices. A CGM provides a substantially continuous estimated blood glucose level through a transcutaneous sensor that measures analytes, such as glucose, in the patient's interstitial fluid rather than their blood. CGM systems typically consist of a transcutaneously-placed sensor, a transmitter and a monitor. A CGM system allows a patient or caregiver to insert a single sensor probe under the skin for multiple days. Thus, the patient is only required to perform a single moderately invasive action with a single entry point in the subdermal layer on, e.g., a weekly basis.

Ambulatory insulin infusion pumps typically allow the patient or caregiver to adjust the amount of insulin delivered, by a basal rate or a bolus, based on blood glucose data obtained by a blood glucose meter or CGM. Some ambulatory insulin infusion pumps may include the capability to interface with a blood glucose meter (BGM) or CGM such as, e.g., by receiving measured or estimated blood glucose levels and prompting the user to adjust the level of insulin being administered or planned for administration or, in cases of abnormally high blood glucose readings, prompting temporary cessation of insulin administration. These portable pumps may incorporate a BGM or CGM within the hardware of the pump or may communicate with a dedicated BGM or CGM via, wired or wireless data communication protocols. Such pumps may be particularly important in facilitating patient compliance and improved or more accurate treatment of diabetes. The delivery of insulin from a portable insulin pump making use of CGM data necessitates accurate and reliable CGM data output.

Generally, when CGM devices are used in conjunction with insulin pumps, the CGM device has a separate display from the insulin pump and the user must manually transfer data from the CGM to the pump in order for the pump to incorporate the data into its functioning. Even in instances where the pump can automatically receive CGM data, such as through a wireless connection, or incorporates the CGM in the pump, a user often must scroll through a number of screens of the pump to review the pump and CGM data and make a determination as to whether and how the data indicates that the user should activate the pump.

Therefore, there is a need for a system and a method for better integrating usage of CGM devices and data with insulin pumps.

### SUMMARY OF THE INVENTION

A portable insulin pump can integrate and display data from a continuous glucose monitor (CGM) to allow a user to more readily determine whether any interaction with the pump is necessary. A screen of the pump, which can be a startup screen automatically displayed whenever the pump is turned on or activated from a sleep mode, can display a current glucose level of the user and historical glucose data obtained from the CGM as well as an estimate of the amount of un-metabolized insulin remaining in the user's body. This information allows the user to immediately assess whether any action needs to be taken with the pump, such as to deliver a bolus, without having to scroll through multiple pages and options to obtain the necessary information.

In an embodiment, a portable insulin pump includes a graphical user interface, a receiver adapted to receive information from a continuous glucose monitor and a processor functionally linked to the receiver and the graphical user interface. The processor can be configured to display a startup screen on the graphical user interface when the graphical user interface is activated from an inactive condition, such as being turned off or in sleep mode. The startup screen can display a current glucose level of a user and historical glucose level data based off of information received from the CGM at the receiver. The startup screen can also display an estimate of the amount of un-metabolized insulin remaining in the user's body.

In another embodiment, a portable insulin pump can integrate with a continuous glucose monitor to automatically calculate a recommended bolus for a user. When the user uses a blood sample to calibrate the CGM with an actual blood glucose reading, the CGM can automatically transmit that blood glucose value to the pump. The pump can then determine whether that value is above a threshold and, if so, automatically calculate a bolus of insulin to bring the user's blood glucose level below the threshold.

Certain embodiments are described further in the following description, examples, claims, and drawings. These embodiments will become more apparent from the following detailed description when taken in conjunction with the accompanying exemplary drawings.

### BRIEF DESCRIPTION OF THE DRAWINGS

FIG. 1 is a perspective view of an infusion pump according to an embodiment of the present invention.

FIG. 2 is a block diagram representing an embodiment of an infusion pump.

FIG. 3 depicts a screen shot of a home screen page of a graphical user interface of an infusion pump according to an embodiment of the present invention.

FIG. 4 is a partial schematic view depicting a continuous glucose monitor according to an embodiment of the present invention deployed on a patient.

FIG. 5 depicts a screen shot of a screen page of a graphical user interface of an infusion pump according to an embodiment of the present invention.

FIG. 6 depicts a screen shot of an unlock screen of a graphical user interface of an infusion pump according to an embodiment of the present invention.

FIG. 7 depicts a screen shot of a bolus recommendation screen of a graphical user interface of an infusion pump according to an embodiment of the present invention.

FIG. 8 is a flowchart of a method of recommending a correction bolus according to an embodiment of the present invention.

### DETAILED DESCRIPTION

Provided herein are systems, devices and methods for integrating usage of ambulatory infusion pumps with continuous glucose monitoring devices. Some embodiments may include advances in the internal components, the control circuitry, and improvements in a user interface of the systems and devices. The advances may allow for a safer and more accurate delivery of medicament to a patient than is currently attainable today from other devices, systems, and methods. Although embodiments described herein may be discussed in the context of the controlled delivery of insulin, delivery of other medicaments, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, or any other suitable indication or application. Non-medical applications are also contemplated.

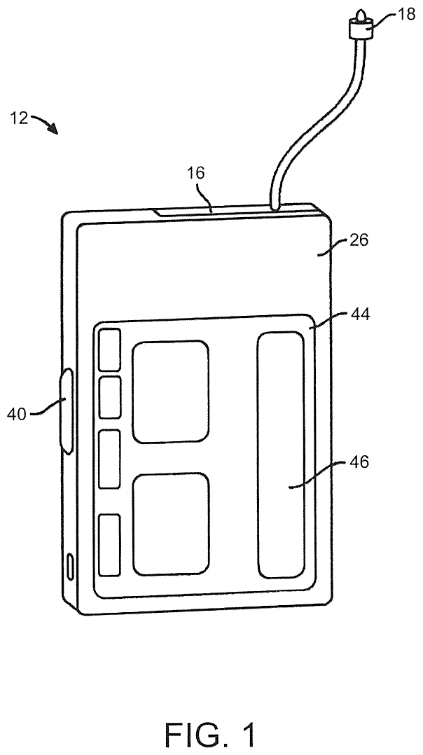

FIG. 1 depicts an embodiment of a pump 12 such as an infusion pump that can include an internal pumping or delivery mechanism and reservoir for delivering medicament such as insulin to a patient and an output/display 44. The type of output/display 44 may vary as may be useful for a particular application. The type of visual output/display may include LCD displays, LED displays, plasma displays, OLED displays and the like. The output/display 44 may also be an interactive or touch sensitive screen 46 having an input device such as, for example, a touch screen comprising a capacitive screen or a resistive screen. The pump 12 may additionally include a keyboard or other input device known in the art for data entry, which may be separate from the display. The output/display 44 of the pump 12 may also include a capability to operatively couple to a secondary display device such as a laptop computer, mobile communication device such as a smartphone or personal digital assistant (PDA) or the like. Further details regarding such pump devices can be found in U.S. Patent Application No. 2011/0144586, which is incorporated herein by reference.

Some embodiments of an infusion system may include a portable infusion device, as described above and a remote commander device. In such an instance, the portable infusion device may include a suitably configured receiver and/or transmitter for communication with an external device such as a remote commander, as well as programming for directing the use of the device; and the remote commander may additionally include a suitably configured receiver and/or transmitter for communication with an external device such as a portable infusion device, as well as programming for directing the use of the device. For instance, the remote commander may include one or more of the functionalities described herein with respect to the portable infusion device. In addition, some GUI embodiments may be available to a user by downloading a software application onto the user's cell phone and/or PDA, which would allow the user to use their cell phone or PDA as a remote commander to the portable infusion device.

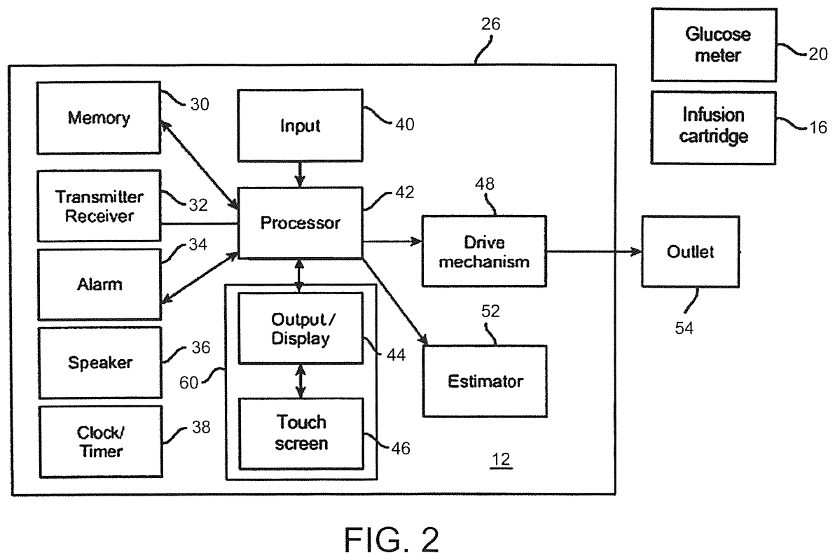

FIG. 2 illustrates a block diagram of some of the features that may be incorporated within the housing 26 of the pump 12. The pump 12 includes a processor 42 that functions to control the overall functions of the device. The infusion pump 12 may also include a memory device 30, a transmitter/receiver 32, an alarm 34, a speaker 36, a clock/timer 38, an input device 40, the processor 42, a user interface suitable for accepting input and commands from a user such as a caregiver or patient, a drive mechanism 48, and an estimator device 52. One embodiment of a user interface as shown in FIG. 2 is a graphical user interface (GUI) 60 having a touch sensitive screen 46 with input capability. The memory device 30 may be coupled to the processor 42 to receive and store input data and to communicate that data to the processor 42. The input data may include user input data and non-user/sensor input data. The input data from the memory device 30 may be used to generate therapeutic parameters for the infusion pump 12. The GUI 60 may be configured for displaying a request for the user to input data and for receiving user input data in response to the request, and communicating that data to the memory.

The processor 42 may communicate with and/or otherwise control the drive mechanism, output/display, memory, a transmitter/receiver and other components. In some embodiments, the processor 42 may communicate with a processor of another device, for example, a continuous glucose monitor (CGM), through the transmitter/receiver. The processor 42 may include programming that can be run to control the infusion of insulin or other medicament from the cartridge, the data to be displayed by the display, the data to be transmitted via the transmitter, etc. The processor 42 may also include programming that may allow the processor to receive signals and/or other data from an input device, such as a sensor that may sense pressure, temperature or other parameters. The processor 42 may determine the capacity of the drug delivery reservoir and/or the volume of fluid disposed in the drug delivery reservoir and may set therapeutic parameters based on its determination.

The processor 42 may also include additional programming to allow the processor 42 to learn user preferences and/or user characteristics and/or user history data. This information can be utilized to implement changes in use, suggestions based on detected trends, such as weight gain or loss. The processor can also include programming that allows the device to generate reports, such as reports based upon user history, compliance, trending, and/or other such data. Additionally, infusion pump device embodiments of the disclosure may include a “power off” or “suspend” function for suspending one or more functions of the device, such as suspending a delivery protocol, and/or for powering off the device or the delivery mechanism thereof. For some embodiments, two or more processors may be used for controller functions of the infusion pumps, including a high power controller and a low power controller used to maintain programming and pumping functions in low power mode, in order to save battery life.

The memory device 30 may be any type of memory capable of storing data and communicating that data to one or more other components of the device, such as the processor. The memory may be one or more of a Flash memory, SRAM, ROM, DRAM, RAM, EPROM and dynamic storage, for example. For instance, the memory may be coupled to the processor and configured to receive and store input data and/or store one or more template or generated delivery patterns. For example, the memory can be configured to store one or more personalized (e.g., user defined) delivery profiles, such as a profile based on a user's selection and/or grouping of various input factors, past generated delivery profiles, recommended delivery profiles, one or more traditional delivery profiles, e.g., square wave, dual square wave, basal and bolus rate profiles, and/or the like. The memory can also store, for example, user information, history of use, glucose measurements, compliance and an accessible calendar of events.

The housing 26 of the pump 12 may be functionally associated with an interchangeable and a removable glucose meter 20 and/or infusion cartridge 16. The infusion cartridge 16 may have an outlet port 54 that may be connected to an infusion set (not shown) via an infusion set connector 18. Further details regarding some embodiments of various infusion pumps can be found in U.S. Patent Application Publication No. 2011/0144586, which is hereby incorporated by reference.

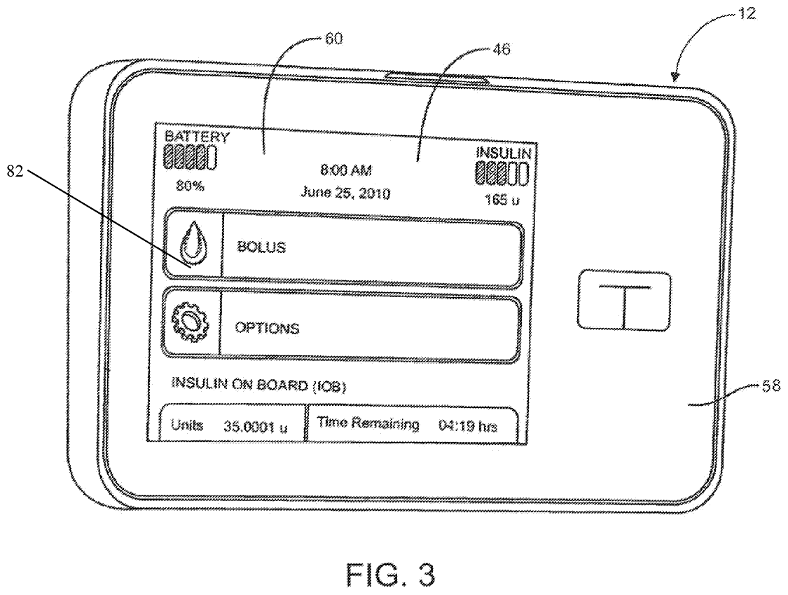

Referring to FIG. 3, a front view of the pump 12 is depicted. The pump 12 may include a user interface, such as, for example, a user-friendly GUI 60 on a front surface 58 or other convenient location of the pump 12. The GUI 60 may include a touch-sensitive screen 46 that may be configured for displaying data, facilitating data entry by a patient, providing visual tutorials, as well as other interface features that may be useful to the patient operating the pump 12. A bolus object 82 can also be displayed on the screen 46.

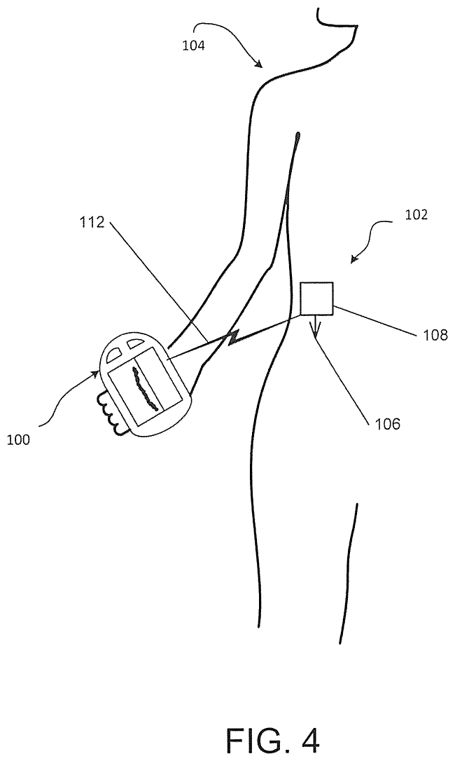

Pump 12 can interface with a continuous glucose monitor (CGM) that provides a substantially continuous estimated glucose level through a transcutaneous sensor that measures analytes, such as glucose, in the patient's interstitial fluid rather than their blood. Referring to FIG. 4, a CGM system 100 according to an embodiment of the present invention is shown. The illustrated CGM system 100 includes a sensor 102 affixed to a patient 104 and is associated with the insulin infusion device 12. The sensor 102 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be calibrated with the patient's blood glucose (BG) level. The sensor 102 includes a sensor body 108 that transmits data associated with the interstitial fluid to which the sensor probe is exposed. The data may be transmitted from the sensor 102 to the glucose monitoring system 100 via a wireless transmitter, such as a near field communication (NFC) radio frequency (RF) transmitter or a transmitter operating according to a “WiFi” or

“Bluetooth” protocol or the like, or the data may be transmitted via a wire connector from the sensor 102 to the monitor system 100. Transmission of sensor data to the glucose monitor system 100 by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In one embodiment, part of the CGM system 100 is incorporated into the pump 12 such that the processor 42 is adapted to receive the data from the sensor 102 and process and display the data on the display 44. In another embodiment, the CGM 100 is a separate device that communicates with the pump 12 through a wired or wireless link to transmit the CGM data to the pump 12.

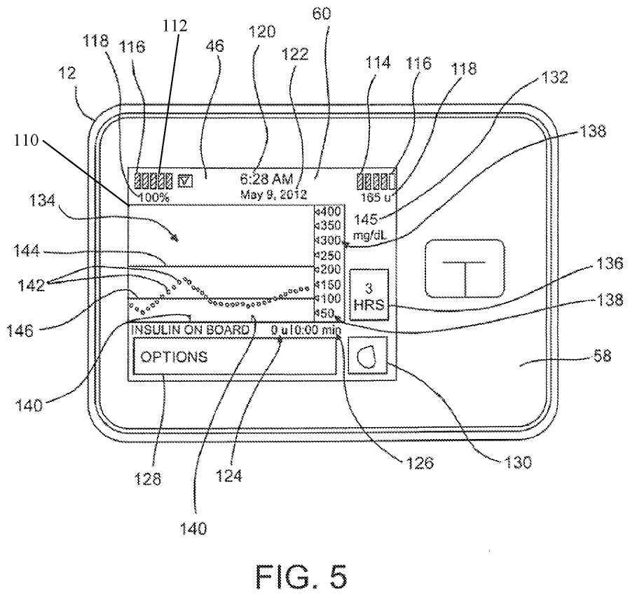

Referring to FIG. 5, there can be seen a pump screen 110 incorporating pump 12 data and CGM data according to an embodiment of the present invention. Pump screen 110 can be displayed on the touch screen 46 of the GUI 60 on the front surface 58 of the pump 12. Screen 110 can include a battery life indicator 112 and an insulin indicator 114. Each indicator 112, 114 can include one or more of a graphical indication, shown in FIG. 4 as a plurality of indicator bars 116 and a textual indicator 118 such as a percentage or amount remaining. The time 120 and date 122 can also be displayed on screen 110. The screen 110 can also include an indication of the amount of insulin on board 124, that is, the amount of un-metabolized insulin already present in the user's body, as well as a time remaining object 126 counting down the amount of time the insulin on board is calculated to remain in the user's body. An options key 128 can allow a user to scroll through various pump operation options and a bolus object 130 can allow a user to begin programming a bolus of insulin.

With further reference to FIG. 5, information regarding the user's glucose level received from the CGM device can be incorporated and displayed on the pump screen 110 alongside insulin pump 12 data. The information can include the user's current glucose level 132 as well as a graphical representation 134 of historical glucose data that can be used, for example, to view glucose level trends and rates of change. A timeframe object 136 can be used to select a timeframe over which to view the historical data. The right side of the graph 134 can include a plurality of glucose values 138 forming the vertical axis of the graph 134. The horizontal axis of the graph 134 tracks the time period over which the data is displayed and can include hash marks 140 or other indicators, including numerical indicators, indicating equal divisions of the selected timeframe. The user's glucose level is therefore shown on the graph 134 as a function of time and can be shown as discrete points 142 as shown in FIG. 5. Alternatively, the glucose level versus time can be shown as a continuous line or curve. The graph 134 can also include a high glucose level indicator line 144 and a low glucose level indicator line 146 identifying predetermined patient specific thresholds that make it easy for a user to see when the user's glucose level has crossed a threshold. Although shown and described as being displayed in a graphical format, CGM data can also be displayed in a textual and numerical format on GUI 60 as well as audibly through, e.g., a speaker.

The pump screen 110 includes all data that the user needs to determine whether interaction with the pump is necessary. For example, for the user to determine whether or not to administer a bolus of insulin, the key information of the user's current glucose level 132, the amount of insulin on board 124 and historical glucose level data 144 indicating recent trends and rates of change is all displayed to the user on one convenient screen. In addition, whether underlying requirements for the pump to deliver a bolus are met, such as the battery life 112 of the pump and the amount of insulin 114 in the pump reservoir, can also be determined from the display of this information on the same screen 110.

The pump screen 110 shown in FIG. 5 can be a default or startup screen of the GUI 60. As a startup screen, pump screen 110 can be the first screen displayed on the GUI anytime the user powers the pump 12 on or activates the pump 12 from a sleep mode. This allows the user to immediately assess whether any action needs to be taken with the pump, such as to deliver a bolus, charge a battery or change a cartridge containing the insulin reservoir. The user can therefore take any necessary action with little more than a glance at the device, rather than having to scroll through multiple pages and options to determine whether action needs to be taken. In some embodiments, the pump 12 can also revert to the startup pump screen 110 whenever the GUI 60 has been inactive for a predetermined period of time and/or whenever a home key or home key sequence is entered.

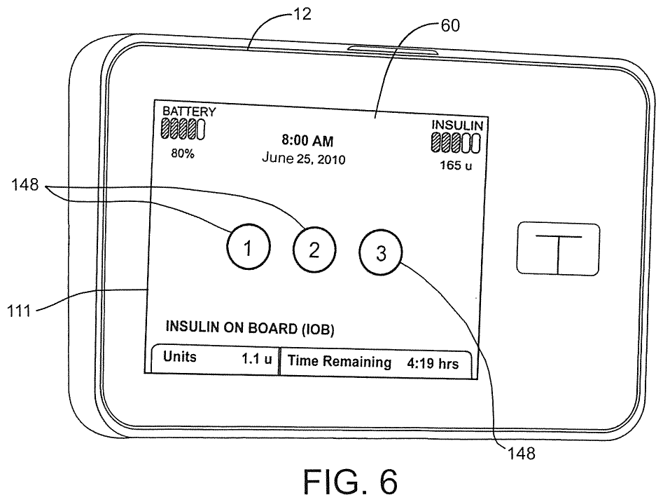

If the user determines from the startup pump screen 110 that a bolus should be delivered, a deliver bolus command can also be begun directly from the screen 110 by selecting the deliver bolus object 130. If some other pump interaction is required, the user can access the necessary command with the options key 128. In some embodiments, before the user can deliver a bolus or otherwise change a pump parameter, the user must unlock the startup pump screen 110, such as by entering a specific numeric sequence or swiping along the touchscreen 46 in a specific manner. An unlock screen such as the unlock screen 111 shown in FIG. 6 can pop up over or in place of the startup pump screen 110 to provide for unlocking of the device. As shown in FIG. 6, in one embodiment unlock screen 111 includes a plurality of numerical indicators 148 with which the user enters a previously saved code to unlock the pump 12. Such an unlock procedure will prevent the user from accidentally altering pump operation when it is not intended. Aspects of these features are further explained in U.S. Provisional Patent Application Ser. No. 61/656,997 and in U.S. patent application Ser. No. 13/801,230 entitled “Preventing Inadvertent Changes in Ambulatory Medical Devices,” filed Mar. 13, 2013 by M. Rosinko, et al., each of which is hereby incorporated by reference.

Because CGM devices estimate blood glucose levels from analyzing interstitial plasma or fluid rather than blood as with blood glucose monitors that utilize a sample of blood obtained from, e.g., a finger stick, CGM devices generally are not as well-suited for accurate blood glucose monitoring. Accordingly, CGMs are most often used for identifying trends in blood glucose levels over time and for providing estimates thereof. However, to ensure that a CGM device is estimating the user's glucose level as accurately as possible, such devices require a user to calibrate with an actual blood sample several times a day that is then used to compare the user's actual blood glucose level with the glucose levels measured by the CGM. Typically, after this is done the user must then manually enter the blood glucose data into the pump to allow therapy parameters to be calculated based on the data. Aspects of advanced calibration techniques that may be used in such systems are found in U.S. patent application Ser. No. 13/841,028 entitled “Predictive Calibration” to Saint, the entirety of which is incorporated by reference.

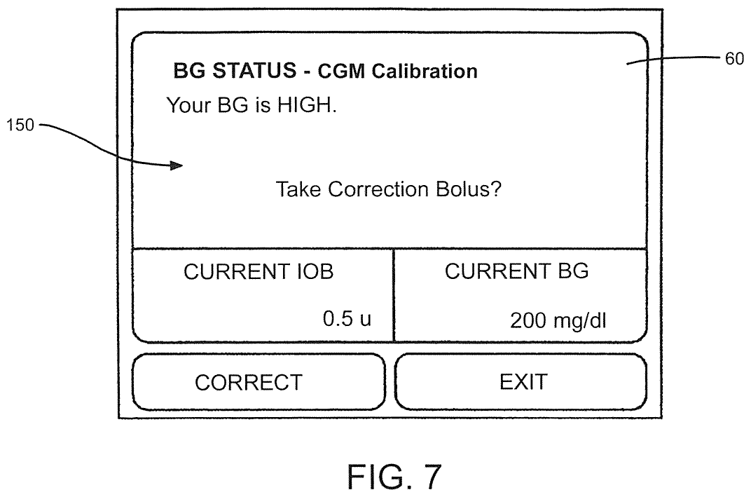

However, in an embodiment of a pump 12 that communicates with a CGM and that integrates CGM data and pump data as described herein, the CGM can automatically transmit the blood glucose data determined from the calibration sample to the pump. The pump can then automatically determine therapy parameters based on the data. For example, if the calibration sample indicates that the user's blood glucose is over a high blood glucose threshold, the pump can automatically calculate an insulin bolus to bring the user's blood glucose below the threshold. In one embodiment, the pump presents the bolus to the user as a recommended bolus 150 on the GUI 60 that the user must approve in order for it to be delivered as shown in FIG. 7. In some embodiments, a visual, audible or tactile alarm, or some combination of these, can automatically be issued to alert the user to a suggested action and reason for the action, such as a high blood glucose reading and corresponding recommended bolus.

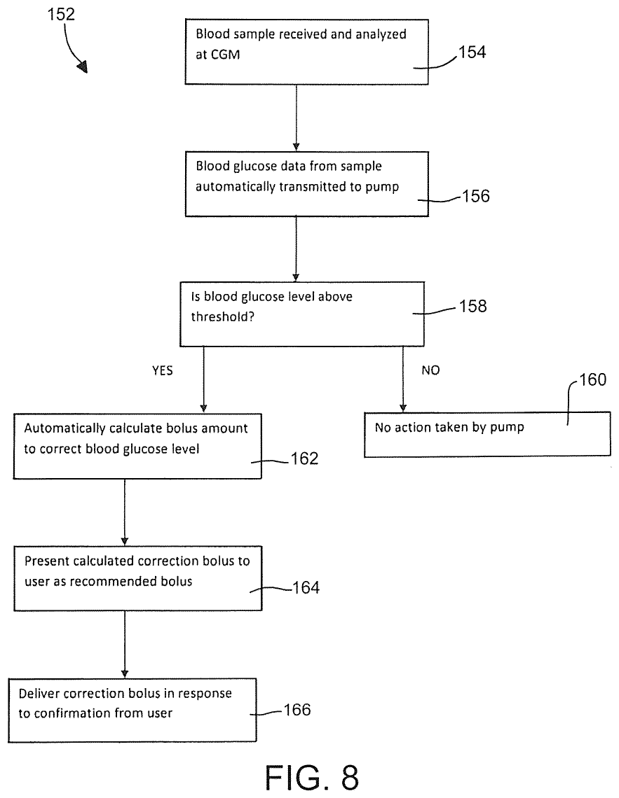

Referring now to FIG. 8, a flowchart of a method of recommending a correction bolus 152 is depicted. At step 154, a user submitted blood sample is received at the CGM for calibration purposes. The blood glucose level of the blood sample is determined and then automatically transmitted to the pump at step 156. The pump determines at step 158 whether the blood glucose level is above a patient specific threshold level. If it is not, at step 160 no action is taken by the pump. If the level is above the threshold, then at step 162 the pump automatically calculates a correction bolus and displays the correction bolus as a recommendation to the user at step 164. If the user confirms the recommended bolus, the bolus is delivered to the user by the pump at step 166.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1-20. (canceled)

21. A method of displaying information to aid in providing diabetes therapy with a portable insulin pump, comprising:
displaying a status screen on a graphical user interface of a portable insulin pump system, the status screen including information relating to glucose levels of the user and a bolus delivery object selectable to initiate a bolus delivery of insulin,
wherein displaying the status screen on the graphical user interface includes displaying the status screen in a locked configuration with the information relating to glucose levels visible but not touch-selectable on the status screen to enable the user to assess whether any action needs to be taken with the portable insulin pump while the status screen is in the locked configuration, and
wherein the bolus delivery object on the status screen is not selectable to initiate a bolus delivery of insulin until the status screen is unlocked from the locked configuration.

22. The method of claim 21, wherein the bolus delivery object is not visible on the status screen when the status screen is in the locked configuration.

23. The method of claim 21, further comprising displaying one or more unlock indicators on the status screen and further comprising receiving user input via the one or more unlock indicators to unlock the status screen from the locked configuration.

24. The method of claim 23, wherein displaying one or more unlock indicators on the status screen includes displaying the one or more unlock indicators over the status screen such that at least a portion of the status screen is visible.

25. The method of claim 21, wherein displaying on the status screen information relating to glucose levels includes displaying one or more glucose level trends.

26. The method of claim 21, wherein displaying on the status screen information relating to glucose levels includes displaying information relating to a rate of change of glucose levels.

27. The method of claim 21, wherein displaying the status screen further includes displaying at least one of a battery life indicator indicating a remaining life of a battery of the portable insulin pump and a reservoir level indicator indicating a remaining amount of insulin in an insulin reservoir of the portable insulin pump.

28. The method of 21, wherein the status screen is displayed upon activating the graphical user interface of the portable insulin pump from an inactive condition.

29. The method of claim 27, wherein activating the graphical user interface from an inactive condition includes activating the graphical user interface from a sleep mode.

30. The method of claim 21, further comprising displaying the status screen when no user input is received through the graphical user interface for a predetermined period of time.

31. A portable insulin pump system, comprising:
a graphical user interface;
and
a processor functionally linked to the graphical user interface to control display of information on the graphical user interface, wherein the processor is configured to display a status screen on the graphical user interface, the status screen including information relating to glucose levels of the user and a bolus delivery object selectable to initiate a bolus delivery of insulin,
wherein the processor is configured to display the status screen in a locked configuration with the information relating to glucose levels visible but not touch-selectable on the status screen to enable the user to assess whether any action needs to be taken with the portable insulin pump while the status screen is in the locked configuration, and wherein the bolus delivery object on the status screen is not selectable to initiate a bolus delivery of insulin until the status screen is unlocked from the locked configuration.

32. The portable insulin pump system of claim 31, wherein the processor is configured to display the status screen with the bolus delivery object not visible when the status screen is in the locked configuration.

33. The portable insulin pump system of claim 31, wherein the processor is further configured to display one or more unlock indicators on the status screen and receive user input via the one or more unlock indicators to unlock the status screen from the locked configuration.

34. The portable insulin pump system of claim 33, wherein the processor is further configured to display the one or more unlock indicators over the status screen such that at least a portion of the status screen is visible.

35. The method of claim 31, wherein displaying on the status screen information relating to glucose levels includes displaying one or more glucose level trends.

36. The method of claim 31, wherein displaying on the status screen information relating to glucose levels includes displaying information relating to a rate of change of glucose levels.

37. The portable insulin pump system of claim 31, further comprising a battery that powers the processor and a reservoir that contains insulin for delivery to the user, and the status screen further includes at least one of a battery life indicator indicating a remaining life of the battery and a reservoir level indicator indicating a remaining amount of insulin in the reservoir.

38. The portable insulin pump system of claim 31, wherein the processor is configured to display the status screen upon activating the graphical user interface from an inactive condition.

39. The portable insulin pump system of claim 38, wherein activating the graphical user interface from an inactive condition includes activating the graphical user interface from a sleep mode.

40. The portable insulin pump system of claim 31, wherein the processor is further configured to display the status screen when no commands have been entered into the graphical user interface for a predetermined period of time.
